(19) United States
(12) Patent Application Publication
(10) Pub. No.: US 2010/0298420 A1
(43) Pub. Date: Nov. 25, 2010

(54) Compositions and Methods for Intracellular Delivery

(75) Inventor: Elka Touitou, Jerusalem (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Yissum, Research Development Company of the Hebrew University of Jerusalem Ltd., Jerusalem (IL)

(21) Appl. No.: 12/793,423

(22) Filed: Jun. 3, 2010

Related U.S. Application Data
(62) Division of application No. 10/482,116, filed on Jul. 13, 2004, now abandoned.
(60) Provisional application No. 60/300,415, filed on Jun. 26, 2001.

(51) Int. Cl. A61K 48/00; C12N 15/09; C12N 15/85; C12N 15/82

(52) U.S. Cl. 514/44 R; 435/440; 435/455; 435/468

(57) ABSTRACT

The invention relates to intracellular delivery of alcoholic lipid compositions for medical, cosmetic, research, diagnostic, veterinary, agriculture or pharmaceutical use containing phospholipid(s), ethanol (or other C2-C4 such volatile alcohols), water, at least one active molecule, optional addition of glycols and/or other additions for delivery to cells of an entrapped, attached, adsorbed, and/or complexed molecule(s).

### FIELD OF THE INVENTION

The invention relates to intracellular deliver alcoholic lipid compositions for medical, cosmetic, research, diagnostic, veterinary, agriculture or pharmaceutical use containing phospholipid(s), ethanol (or other C2-C4 such volatile alcohols), water, at least one active molecule, optional addition of glycols or/and other additions for delivery to cells of an entrapped, attached, adsorbed, complexed molecule(s).

### BACKGROUND OF THE INVENTION

The cell membrane plays a crucial role in physiological homeostasis, allowing selected molecules to penetrate while preventing the permeation of others. Breaking down the permeability barrier, however, can be useful when delivery of otherwise impermeant active agents is desired. Whether for pharmaceutical purposes, gene therapy, vaccination, delivery to microorganisms or cellular transformations in biomedical research or for agricultural use to vegetal cells the delivery of molecules intracellulary has become a major focus of research in recent years. The use of lipid vesicular systems is one method that has been used to overcome this obstacle of penetration. While classic liposomes are unable to improve the penetration of impermeable molecules through the cell membrane barrier, some specially designed lipid vesicles were shown to efficiently deliver their contents to the cytoplasm.

Several approaches have been described to improve intracellular delivery by vesicular systems. One of these approaches involves increasing the encapsulation efficiency of molecules by imparting a charge to the lipid vesicles. Liposomes containing mono-cationic lipids have been used to transfect cells with DNA or RNA in vitro and in vivo (Wrobel and Collins, 1995), as well as to increase the uptake of other impermeable agents (Garrett et al., 1999). Cationic liposomes that can undergo lipid mixing with cellular membranes were reported to deliver complexed DNA to cells, most likely via an endocytotic process (Miller et al. 1998). Polycationic liposomes were shown to enhance delivery of β-galactosidase and human placental alkaline phosphatase to various cell cultures (Sells et al, 1995). Another approach involved modifying the lipid composition of vesicles, for example, by incorporating steric stabilizers such as PEG (Duzgunes and Nir, 1999; Miller et al 1998). Other attempts to affect the intracellular fate of encapsulated molecules focused on pH-sensitive liposomes (Chu et al., 1990; Kono et al., 1997). Co-administration of liposomes with dimethyl sulfoxide was also found to improve delivery by some vesicular systems (Jain and Gewirtz, 1998; Kawai and Nishizawa, 1984).

European patent 0 804 160 and U.S. Pat. No. 5,716,638 disclose systems (Ethosomes) that were found to be highly efficient carriers for the delivery of molecules with various lypophilicities into and through the skin. The main route of molecules penetration in the skin is intercellular (between cells) and not transcellular.

Thus, there is a need to a composition that is easy to prepare, that will improve cellular uptake and trafficking, will enable delivery of agents to cells, glands, tissues and organs and in another embodiment, will enable the delivery to the cell's nucleus or other cellular organelles.

### BRIEF DESCRIPTION OF THE DRAWINGS

FIG. 1 demonstrates CLSM micrograph showing intracellular fluorescence in fibroblasts following delivery of fluorescent probes from Compositions and control systems.

A-1, A-2, A-3: delivery from compositions I, II and III, respectively;
B-1, B-2, B-3: delivery from control liposomal system (control B);
C-1, C-2, C-3: delivery from control hydroethanolic solution (control A).

FIG. 2 demonstrates CLSM micrograph showing intracellular fluorescence in 3T3 fibroblasts following delivery of fluorescent Phosphatidylcholine (PC*, see examples) from Compositions containing organic cations and control systems: liposomes (a), Formulation 1 (b), Propranolol formulation (c) and THP formulation (d).

FIG. 3 demonstrates CLSM micrograph showing intracellular fluorescence in 3T3 fibroblasts following delivery of Rhodamine red labeled phospholipid (RR, see examples) from: a—Composition containing organic cation (THP) and control systems (b—hydroethanolic solution, c—liposomes)

FIG. 4 demonstrates CLSM micrograph showing intracellular fluorescence of secondary antibody following transfection of fibroblasts with p53 plasmid by using composition VII.

FIG. 5 demonstrates CLSM micrograph showing GFP intracellular expression, following transfection of whole tissue (skin) with CMV-GFP cDNA delivered from Composition VIII (M2) vs. Control (M1).

### DESCRIPTION OF THE DETAILED EMBODIMENTS

This invention relates to a method and a hydro-alcoholic or hydro/alcoholic/glycolic lipid composition containing at least a phospholipid, ethanol (or other C2-C4 volatile alcohols), water for the penetration through biological membranes and for the facilitation of the delivery of entrapped or complexed molecules through biological and cellular membranes, into cells and cellular organelles such as for example the cell nucleus.

In another embodiment, the composition further comprises organic small cation.

In another embodiment the composition may contain a small molecular weight cation, which refers hereinafter to a organic cationic molecule with a molecular weight of 100-600.

In another embodiment the composition may contain a small molecular weight cation, which refers hereinafter to an organic cationic molecule which is not phospholipid.

The composition and the method of the invention can be used for pharmaceutical, cosmetic, medical, veterinary, diagnostic, agriculture and research applications.

The advantages of the method and the composition of the invention are as follows:

Improved cellular uptake and trafficking.
The composition is easy to prepare.
Delivery into cells, tissues, glands, follicles and organs.
Delivery to nucleus (or other cellular organelles).

In one embodiment, the composition may contain phospholipid, ethanol, water and non-phospholipid organic amphiphilic cation for the penetration through biological membranes and for the facilitation of the delivery of entrapped or complexed molecules through biological and cellular membranes, into cells and cellular organelles.

The presence of ethanol, in an amount of 10 to 50% provides a negative charge to the vesicle. The incorporation of the positive ions to such compositions provides a vesicle with a positive charge.

In another embodiment, the composition may contains also other volatile C2-C4 alcohols.

In another embodiment, the composition may include another C2-C4 volatile alcohol instead of the ethanol.

The composition comprises a phospholipid, more than 10% ethanol (or other C2-C4 volatile alcohols), from 0 to 30% glycols and water.

In another embodiment, the composition of the invention may also contain 0 to 40% polyols.

In another embodiment the composition may comprise phospholipids, ethanol (EtOH), water (DDW), and propylene glycol (PG).

In another embodiment, cationic composition may be prepared in addition to the phospholipid, more than 10% ethanol (or other C2-C4 volatile alcohols), from 0 to 30% glycols and water, non-phospholipidic cationic amphiphilic molecules. The non-phospholipidic cationic amphiphilic molecules of the invention are relatively small molecular weight (MW 100-600) that do not belong to the group of phospholipids, such as, for example without being limited, propranolol HCl.

In another embodiment, the composition can be used for the delivery of agents to cells (which can be also in culture), membranes, glands, hair follicles, hair shafts, sebaceous glands, tissues, or whole organs of plants or animals, either in vitro, ex vivo or in vivo. The composition may penetrate through biological and cellular membranes and facilitates the penetration of entrapped or complexed molecules through these membranes.

The composition of the invention may contain different phospholipids, such as without being limited, phosphatidylcholine (PC), hydrogenated PC, phosphatidic acid (PA), phosphatidylserine (PS), phosphatidylethanolamine (PE), phosphatidylglycerol (PPG), phosphatidylinositol (Pl), their mixture, cationic phospholipids, ceramides and other lipids. In addition the composition may contain other additives such as cholesterol, surfactants and others.

Phospholipids are known for their broad use in liposomal systems as well as emulsifiers in the preparation of emulsions. All these systems used for pharmaceutical or cosmetic purposes are aqueous systems with small if any concentration of alcohol and/or glycol for preservation and/or improving texture of the formulation.

The source of the phospholipids can be egg, soybean, semi-synthetics, and synthetics.

The concentration of alcohol (EtOH etc.) in the final product ranges from about 10-50%. The concentration of the non-aqueous phase (alcohol and glycol combination) may range between about 12 to 70%. The rest of the carrier contains water and possible additives.

The compositions can effectively deliver molecules intracellulary.

The molecule which can be delivered by the composition of the invention is, without being limited, antimicrobial agent, antiparazitic, insecticide, therapeutic agent, chemotherapeutic agent, biological tools, diagnostic agent, peptide antibiotic, antiacne agent, mitotic, antimitotic, steroid, antihirsutism, agent for hair growth hormone, vitamin, antibiotic, antifungal, antiviral, nucleic acids (DNA, RNA, plasmids), proteins/peptides/aminoacids, lipids, sugars, glycoproteins, glycolipids, antisense oligonucleotides (ODNs), polyanionic macromolecules and derivatives, nucleic acids, ON's, DNA and RNA oligonucleotides, naked ODNs, vitamins, antibiotics, various macromolecules.

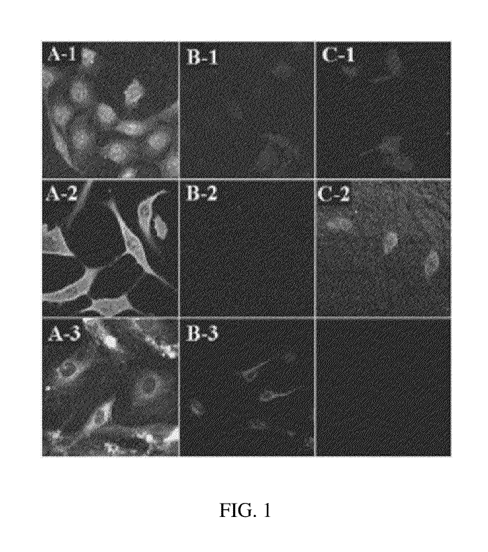

As is shown in FIGS. 1-4 the compositions of the invention can effectively deliver molecules through membrane into the cell cytoplasm.

The composition can effectively deliver molecules to the nucleus of cells and/or other organelles as described in Example 1 in the Examples section.

The composition can effectively deliver molecules into microorganisms, microbes, pathogens and the like.

The compositions can be administered IP, IM SC Iv intratumor or interdermal. The composition may be in a form of solid, liquid spray, patch or semi liquid.

In another embodiment the composition may be administered in iontophoresis, phonophoresis, microporation, microneedles, electroporation, jet, laser.

In another embodiment, the composition is added to a culture in a quantity of 10-200 ul/well, wherein the well volume is 1-2 ml. The same ratio is maintained also in other sizes of wells.

The composition of the invention, can be administered to any part of the plant. i.e. leaves, roots, cortex, stem, earth, flowers, buds.

When used in gene therapy, the compositions of the invention may contain non-phospholipid organic cation to be used to deliver DNA into the selected eukaryotic cell. Protocols for stable transformation and expression of DNA integrated into the genome of the transfected cell are known. Typical protocols for liposome-mediated transfections are described in Ausebel et al. Current Protocols in Molecular Biology, Volume 1, Unit 9.4.1 and, also generally, see Chapter 9 for Introduction of DNA into Mammalian Cells. The ability of the composition of the invention to facilitate cell transfection is demonstrated in Example 4.

The nucleic acid compositions of this invention, whether nuclear RNA, mRNA, cDNA, genomic DNA, plasmid DNA, or a hybrid of the various combinations, are isolated from biological sources (including recombinant sources) or synthesized in vitro. The nucleic acids of the invention are present in transformed or transfected whole cells, in transformed or transfected cell lysates, or in a partially purified or substantially pure form; when complexed to lipids, the nucleic acids are typically in substantially pure form.

Nucleic acids which can be used for inclusion in the complexes of the invention include those with therapeutic relevance to cancer. For example, nucleic acids which inhibit expression of oncogenes such as HER-2/neu (e.g., the tumor suppressor E1A from adenovirus 5), or which control cell growth or differentiation are preferred components of the lipid:nucleic acid complexes of the invention. For example, nucleic acids which encode expression of cytokines, inflammatory molecules, growth factors, telomerase, growth factor receptors, oncogene products, interleukins, interferons, .alpha.-FGF, IGF-I, IGF-II, .beta.-FGF, PDGF, TNF, TGF-.alpha., TGF-.beta., EGF, KGF, SCF/c-Kit ligand, CD40L/CD40, VLA-4/VCAM-1, ICAM-1/LFA-1, and hyalurin/CD44; signal transfection molecules and corresponding oncogene products, e.g., Mos, Ras, Raf, and Met; and transcriptional activators and suppressors, e.g., p53, p21, Tat, steroid hormone receptors such as those for estrogen, progesterone, testosterone, aldosterone, and corticosterone or the like are known, preferred, and widely available. Nucleic acids which encode inhibitors of such molecules are also preferred, such as ribozymes and anti-sense RNAs which recognize and inhibit translation of the mRNA for any of the above. Finally, nucleic acids encoding suicide genes which induce apoptosis or other forms of cell death are preferred, particularly suicide genes which are most active in rapidly dividing cells (e.g., cancer cells), such as the herpes simplex virus thymidine kinase gene in combination with gancyclovir, the E1A gene product from adenovirus, or a variety of other viral genes. Negative selectable markers which are not activated until a counter agent is added are also appropriate. Decoy nucleic acids which encode molecules that bind to factors controlling cell growth are appropriate to some applications. Nucleic acids encoding transdominant molecules are also appropriate, depending on the application.

The compositions of the invention can also be used to introduce nucleic acid, e.g. plasmid DNA into protoplasts of prokaryotic cells by methods known in the art.

The compositions of the invention can be used to introduce nucleic acids into protoplasts of plant cells. Phospholipids vesicles have been used for intracellular delivery of liposomal contents into plant cells in reported work with tobacco protoplasts. Tobacco mosaic virus (TMV), RNA has been encapsulated in liposome preparations using the reverse evaporation method developed by Szoka and Papahadjopoulos. See PNAS USA 75:4194-4198 (1978). Studies with a variety of plant species (flower and vegetable), like tomato, lily, daylily, onion, peas, petunia and others have been reported. See, Genetic Engineering of Plants, Ed. Kosuge, Merideith and Hollaender, published by Plenum Press, authored by Fraley and Horsch, entitled “In vitro Plant Transformation Systems Using Liposomes and Bacterial Co-Cultivation”, Vol. 26, pps. 177-194 (1983) and other articles therein. In a similar manner, the compositions of the invention with appropriate adaptation by one skilled in the art to best fit the purpose intended, can be used to transform plants.

The composition of the invention which comprises as an active agent a DNA plasmid is suitable for direct injection into the tumor lesion of a patient. Such a composition can be applied as an aerosol into the airways, such as the trachea, the nasal or other cavities of a cystic fibrosis patient. Likewise, such a composition may be contemplated for peritonial injection into a patient with ovarian carcinoma with metastasis in the peritonial cavity. For the treatment of neurological diseases of Alzheimer disease, direct injection and transfection of brain cells to cause express of a therapeutic copy of the defective target gene is of major interest. The compositions of the invention are likewise considered useful for gene therapy of muscular dystrophy, hemophilia B and several other diseases caused by defective genes. The composition may contain one or more of the cationic molecule of the invention. It is not excluded to use other cationic molecule with one or more cationic molecule of the invention, providing the formulation is adequately stable and effective for cell transfection. One skilled in the art with the knowledge of the properties of the cationic molecules of the invention (and with the knowledge of the other lipids) can readily formulate a composition best suited for the particular cell transfection desired.

In another embodiment the composition is typically mixed with polyanionic compounds (including nucleic acids) for delivery to cells. Complexes form by charge interactions between the cationic compositions and the negative charges of the polyanionic compounds. Polyanions of particular interest include nucleic acids, e.g., DNA, RNA or combinations of the two. Neutral lipids are optionally added to the complex.

In another embodiment the invention provides a method of delivering of an agent into a cell by administering the composition of invention.

In another embodiment the invention provides a method of delivering a nucleic acid sequence into a nucleus of a cell by administering the composition of the invention.

In another embodiment, the invention provides a method of delivering a nucleic acid sequence into a nucleus of a cell by administering the composition of the invention.

In order to facilitate a further understanding of the present invention, the following Examples are given primarily for the purposes of illustrating certain more specific details thereof.

### EXAMPLES

### Example 1

### Intracellular Delivery of Fluorescent Probes

### Experimental Procedures

Materials:

Rhodamine red dihexadecanoylglycerophosphoethanolamine (RR), 4-(4-diethylamino)styryl-N-methylpyridinium iodode (D-289), calcein and the live/dead viability/cytotoxicity kit were purchased from Molecular Probes (Eugene, Oreg., USA). Fluorescent phosphatidylcholine [1-palmitoyl-2-[12-7-nitro-2-1,3-benzoxadiazil 1-4 yl amino[dodecanyl]sn-glycero-3]]-phosphatidylcholine (PC*) was from Avanti Polar Lipids (USA). Phospholipon90 was from Natterman GMBH (Germany). Ethanol was from Frutarom (Israel). Dulbecco's Modified Minimal Essential Medium (DMEM) and Dulbecco's Phosphate Buffered Saline (PBS) were from Biological Industries, Beit HaFinek Israel. All other materials were of analytical grade.

Preparation of the Compositions:

Phospholipon 90 (PL) and probe (0.03% w/w) were dissolved in ethanol. In the experiments of which small cationic molecules (such as propranolol HCl or trihexyphenidyl HCl (THP) were added, the compound was also dissolved in the ethanolic phase. Water was added in aliquots (to the desired concentration), while mixing in a Heidolph digital 2000 RZR-2000 (Heidolph Digital, Germany). Liposomes were prepared by the classic composition method. Briefly, PL and fluorescent probe were dissolved in chloroform, followed by evaporation of the solvent using an R-rotary evaporator (Buchi, Germany) and hydration of the thin film remaining on the inner wall of the flask.

The following abbreviations are used in this application:
PC*—phosphatidylcholine [1-palmitoyl-2-[12-7-nitro-2-1,3-benzoxadiazil 1-4 yl amino[dodecanyl]sn-glycero-3]]-phosphatidylcholine
PL—Phospholipon 90 (egg phosphotidylcholine)
RR—Rhodamine red dihexadecanoylglycerophosphoethanolamine
D-289—4-(4-diethylamino)styryl-N-methylpyridinium iodode
DDW—double distilled water
EtOH—ethanol
THP—trihexyphenidyl (as HCl or at a pH when the molecule is ionized)
Propranolol—as HCl or at a pH when the molecule is ionized

### Composition I:

Two grains of PL and 0.03 g of D-289 were dissolved in 3 g ethanol. DDW was added in aliquots to 10 g, by mixing in a Heidolph digital 2000 RZR-2000.

### Composition II:

The method is the same as Composition I, PC* is used instead of D-289.

### Composition III:

The method is the same as Composition I, RR is used instead of D-289.

### Control Systems:

A) Hydroethanolic solution of the probe: 0.03 g of the probe (RR or D-289 or calcein) was dissolved in 3 g ethanol and complete to 10 g with DDW.
B) Liposomes: liposomes were prepared by classic dispersion method (New, 1990). Briefly, PL and fluorescent probe were dissolved in chloroform (Frutarom, Israel), the solvent was evaporated using an R-rotary evaporator (Buchi, Germany) and the thin film remaining on the inner wall of the flask was hydrated with DDW.
C) System containing no probe: 2 g of PL was dissolved in 3 g ethanol. The DDW was added in aliquots to 10 g, while mixing in a Heidolph digital 2000 RZR-2000.

### Cell Culture:

Subconfluent Swiss albino mouse fibroblast cells (3T3) were grown in Dulbeco's Modified Eagles Medium (DMEM) on coverslips in wells of 3.5 cm in diameter for Confocal Laser Scanning Microscopy (CLSM) and in six-well plastic plates for flow cytometric analysis.

### CLSM Experiment:

The cells were washed twice with phosphate buffered saline (PBS), adjusted to 37° C. in the incubator, and washed again. Two ml of PBS were added to each well and 50 μl of the test solution was added (Compositions I-III, control systems A-B or any compositions containing PC*, RR or D-289 listed above). Cells were incubated with in a presence of test formulations for 0, 3, 7, 10 or 30 minutes. Following incubation, the medium was removed, the cells were fixed for 3 min with 1 ml methanol, and were washed twice with PBS. The coverslip were observed under a Sarastro-Phibos1000 CLS microscope equipped with a 488 nm argon ion laser beam and attached to a Universal Zeiss epifluorescence microscopy with an oil immerse Planapo 63×1.4 NA objective lens. Fluorescence emission was detected above 560 nm for RR, at 527 nm for D-289 and at 488 nm for calcein.

### Flow Cytometry

The cells were washed twice with phosphate buffered saline (PBS), adjusted to 37° C. in the incubator, and washed again. Two ml of PBS were added in each well and 50 μl of the test solution was added (Composition I or control system C). Cells were incubated with gentle shaking in a presence of test systems for 0, 3, 7, 10 or 30 minutes. After the incubation, the medium was removed and the cells were trypsinized (37° C., 2 min). The cells were further treated with 1.5 ml PBS with 10% fetal calf serum (FCS) and were collected in tubes. Following centrifugation (1000 rpm, 5 min), the supernatant was removed and the cells were fixed with formaldehyde. The cells were resuspended in 300 μl of PBS to a final concentration of 0.5×10⁶. Flow cytometric analysis for D-289 fluorescence was performed using a four-color FACS scan (Becton-Dickinson Immunocytometry Systems, USA) and LysysII software. For each analysis 50,000 to 200,000 gated events were collected. D-289 fluorescence was collected on a logarithmic scale with 1024 channel resolution. The mean fluorescence intensity values was determined as linear values from LysysII software.

### Experimental Results

Time-dependent penetration of the amphiphilic fluorescent dye D-289 from Compositions II was measured by CLSM. Maximum penetration (27.5±1.2 arbitrary units), as determined by fluorescence intensity, was reached within 10 minutes, and stayed constant for at least 20 minutes. The fluorescence level of control system C, not containing probe, did not change throughout the experiment. Delivery to fibroblasts of D-289, encapsulated in Composition I, was also assessed by FACS. A 10 minute delivery time, which was shown to represent a plateau level, was used to measure delivery by FACS. The initial (t=0 min) mean fluorescent intensity (MFI) was found to be 22.47±11.56 and increased to 474.60±68.24 after 10 minutes. It is noteworthy that in both FACS and CLSM experiments, penetration of the probe was observed within 3 minutes of incubation.

The delivery of three different probes D-289 and RR, and PC*, from Compositions I-III and control systems A-B was examined as well (FIG. 1). Ten minutes after application, fluorescence was only observed in cells that had been treated with Compositions I, II, III and not the control systems. Penetration of fluorescent lipids (RR and PC*) indicated that the components of those systems themselves penetrated the fibroblasts. For the probe D-289, fluorescence was also observed in the nucleus of the cell as well.

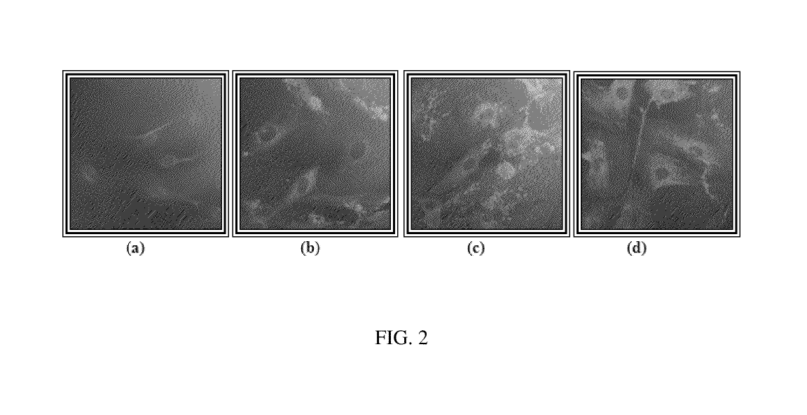

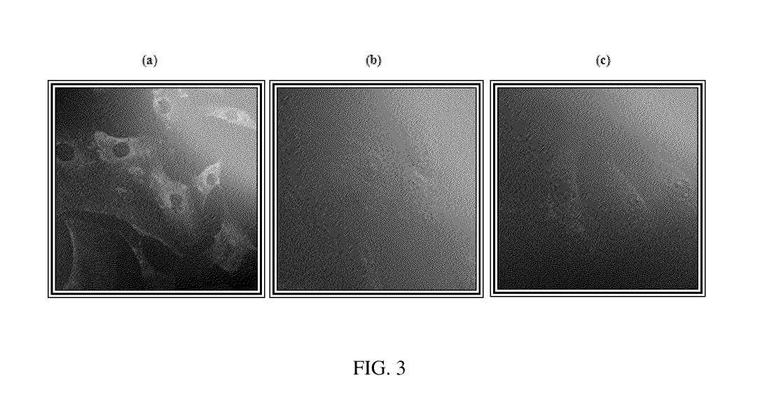

When fluorescent probes were delivered from systems containing small organic cations, the level fluorescence observed was much more intense, as well as a high level of fluorescence observed within the nucleus of the cell. Those findings are demonstrated in FIGS. 2 and 3.

Formulations Prepared with PC* (FIG. 2):

Formulation 1

| | % w/w |
|---|---|
| PC* | 0.03% |
| PL | 2% |
| EtOH | 30% |
| DDW to | 100% |

Propranolol Formulation:

| | % w/w |
|---|---|
| PC* | 0.03% |
| PL | 2% |
| Propranolol | 1% |
| EtOH | 30% |
| DDW to | 100% |

THP Formulation

| | % w/w |
|---|---|
| PC* | 0.03% |
| PL | 2% |
| THP | 1% |
| EtOH | 30% |
| DDW ad | 100% |

Other Formulations with PC* Containing Propranolol:

| | % w/w | | | |
|---|---|---|---|---|
| PL | 2% | 1% | 5% | 10% |
| PC* | 0.03% | 0.03% | 0.03% | 0.03% |
| Propranolol | 0.1% | 0.2% | 0.15% | 0.3% |
| EtOH | 30% | 20% | 40% | 10% |
| DDW to | 100% | 100% | 100% | 100% |

Other Formulations with PC* Containing THP:

| | % w/w | | | |
|---|---|---|---|---|
| PL | 2% | 1% | 5% | 10% |
| PC* | 0.03% | 0.03% | 0.03% | 0.03% |
| THP | 0.1% | 0.2% | 0.15% | 0.3% |
| EtOH | 30% | 20% | 40% | 10% |
| DDW to | 100% | 100% | 100% | 100% |

Formulations Prepared with RR (FIG. 3):

| | % w/w |
|---|---|
| RR | 0.03% |
| PL | 2% |
| THP | 1% |
| EtOH | 30% |
| DDW ad | 100% |

Examples of Formulations with RR Containing Propranolol:

| | % w/w | | | |
|---|---|---|---|---|
| PL | 2% | 1% | 5% | 10% |
| RR | 0.03% | 0.03% | 0.03% | 0.03% |
| Propranolol | 0.1% | 0.2% | 0.15% | 0.3% |
| EtOH | 30% | 20% | 40% | 10% |
| DDW to | 100% | 100% | 100% | 100% |

Other Formulations with RR Containing THP:

| | % w/w | | | |
|---|---|---|---|---|
| PL | 2% | 1% | 5% | 10% |
| RR | 0.03% | 0.03% | 0.03% | 0.03% |
| THP | 0.1% | 0.2% | 0.15% | 0.3% |
| EtOH | 30% | 20% | 40% | 10% |
| DDW to | 100% | 100% | 100% | 100% |

Formulations Prepared with D-289:

Formulation 3

| | % w/w |
|---|---|
| D-289 | 0.03% |
| PL | 2% |
| EtOH | 30% |
| DDW ad | 100% |

Propranolol Formulation:

| | % w/w |
|---|---|
| D-289 | 0.03% |
| PL | 2% |
| Propranolol | 1% |
| EtOH | 30% |
| DDW to | 100% |

Other Formulations with D-289 Containing Propranolol:

| | % w/w | | | |
|---|---|---|---|---|
| PL | 2% | 1% | 5% | 10% |
| D-289 | 0.03% | 0.03% | 0.03% | 0.03% |
| Propranolol | 0.1% | 0.2% | 0.15% | 0.3% |
| EtOH | 30% | 20% | 40% | 10% |
| DDW to | 100% | 100% | 100% | 100% |

THP Formulation

| | % w/w |
|---|---|
| D-289 | 0.03% |
| PL | 2% |
| THP | 1% |
| EtOH | 30% |
| DDW ad | 100% |

Other Formulations with D-289 Containing THP:

| | % w/w | | | |
|---|---|---|---|---|
| PL | 2% | 1% | 5% | 10% |
| D-289 | 0.03% | 0.03% | 0.03% | 0.03% |
| THP | 0.1% | 0.2% | 0.15% | 0.3% |
| EtOH | 30% | 20% | 40% | 10% |
| DDW to | 100% | 100% | 100% | 100% |

### Example 2

### Effect of Delivery Systems on Viability of the Cultured Cells

### Experimental Procedures

### Live/Dead Viability/Cytotoxicity Test:

The intracellular esterase activity and cell membrane integrity was detected using the live/dead viability/cytotoxicity kit (Molecular Probes, Molecular Probes, Eugene, Oreg., USA). The cells were incubated with various test systems as previously described. Test solution were prepared from ethidium homodimer (20 μl), 10 ml PBS and calcein solution. At the end of the incubation, the medium was removed and 1 ml test solution was added to the wells. The plates were left at room temperature for 30 minutes. Cover slips were removed from the plates and observed under the CLSM as described above.

### Experimental Results

This experiment was conducted in order to determine whether the penetration of fluorescent probe described above was due to penetration enhancement rather than loss of cellular viability. Cultured cells were tested for membrane integrity and viability by using the Live/dead viability/cytotoxicity test. This test is based on a) the reaction of Calcein with intracellular esterases and b) reaction of ethidium homodimer with nucleic acids through damaged membranes. Development of green color indicates viability, while the red color indicates dead cells. The results of this test clearly demonstrated that the treated cells are viable following application of the various formulations.

### Example 3

### Susceptibility Testing

### Composition IV:

Erythromycin (Trima, Israel) stock was prepared in ethanolic solution. 0.2 g of PL (Phospholipon90, Natterman GMBH, Germany) was dissolved in 2 g ethanol (Frutarom, Israel) desired volume of ethanolic stock solution of the drug was added to achieve final concentration and completed to 3 g with ethanol 6.8 g of DDW was added in aliquots, by mixing in a Heidolph digital 2000 RZR-2000 (Heidolph Digital, Germany). The preparation sterilize by passing through the 0.2μ filter.

### Composition V:

Erythromycin (Trima, Israel) stock was prepared in ethanolic solution: 0.2 g of PL (Phospholipon90, Natterman GMBH, Germany) and 0.1 g of N-decylmethylsulfoxide (Division Alameda Laboratories, Los Angles, USA) were dissolved in 2 g ethanol (Frutarom, Israel). The desired volume of ethanolic stock solution of the drug was added and completed to 3 g with ethanol 6.7 g of DDW in aliquots was added, by mixing in a Heidolph digital 2000 RZR-2000 (Heidolph Digital, Germany). The preparation was sterilize by passing through the 0.2μ filter.

### Standards:

Various concentrations of erythromycin in sterile water (1.25, 7.5, 10, 12.5 μg/ml) The bacterial strains used were:

Bacillus Subtilis ATCC-6633

Staphylococcus Aureus ATCC-29213

Staphylococcus Aureus erythromycin resistant (clinical strain)

Each erythromycin concentration was mixed with Composition I, Composition II and Standard solution with phosphate buffer saline (PBS, Biological Industries, Beit HaEmek Israel 1:1 by volume. 20 μl of Compositions I-II and standards containing various antibiotic concentrations was added to 6 mm wells on Petri plates containing Tryptic Soy Agar (TSA) inoculated with the microorganisms. The preparation was incubated aerobically at 37° C. for 24 h. The zone of inhibition for each sample was measured.

### Experimental Results

### Bacillus Subtilis ATCC-6633

| Zone of inhibition (mm): | |
|---|---|
| Erythromycin conc. 1.25 microgram/ml | |
| Standard | 7.83 ± 0.26 |
| Composition IV | 10.75 ± 0.42 |
| Composition V | 10.92 ± 0.20 |
| Erythromycin conc. 7.5 microgram/ml | |
| Standard | 13.58 ± 0.38 |
| Composition IV | 18.92 ± 021 |
| Composition V | 18.75 ± 0.42 |

### Staphylococcus Aureus ATCC-29213

| Zone of inhibition: | |
|---|---|
| Erythromycin conc. 1.25 microgram/ml | |
| Standard | 0 |
| Composition IV | 8.5 ± 0.45 |
| Composition V | 10.17 ± 0.41 |
| Erythromycin conc. 10 microgram/ml | |
| Standard | 13.67 ± 026 |
| Composition IV | 16.33 ± 0.41 |
| Composition V | 17.92 ± 0.20 |

### Staphylococcus Aureus Erythromycin Resistant (Clinical Strain)

| Zone of inhibition (mm): | |
|---|---|
| Erythromycin conc. 10 microgram/ml | |
| Standard | 0 |
| Composition IV | 8.25 ± 0.27 |
| Composition V | 10.33 ± 0.41 |
| Erythromycin conc. 12.5 microgram/ml | |
| Standard | 7.67 ± 0.26 |
| Composition IV | 9.30 ± 0.26 |
| Composition V | 11.85 ± 0.26 |

### Example 4

### Intracellular Gene Delivery In Vitro

### Composition VI:

Stock solution was prepared by dissolving 0.2 g PL in 2.5 g ethanol and than adding 6.3 g DDW in aliquots by mixing (Heidolph digital 2000 RZR-20000). Before the beginning of experiment (15 min), 180 microliters of stock solution (containing 4 mg PL) were added in aliquots to 20 microliters of aqueous solution containing 6 micrograms of EGFP cDNA and were shaked gently. Final cDNA concentration was 6 microgram/200 microliter.

### Composition VII:

Stock solution: 0.2 g PL were dissolved in 2.5 g ethanol, 6.3 g DDW was added in aliquots by mixing (Heidolph digital 2000 RZR-2000). Before the beginning of experiment (15 min) 180 microliters of stock solution were added in aliquots to 20 microliters of aqueous solution containing 6 micrograms p53 cDNA and shaked gently. Final cDNA was concentration 6 microgram/200 microliter.

### Composition VIII:

Stock solution: dissolve 0.2 g PL in 2.5 g ethanol and than add 6.3 g DDW in aliquots mixing (Heidolph digital 2000 RZR-20000). Before the beginning of experiment (15 min) 180 microliters of stock solution (containing 4 mg PL) were added in aliquots to 20 microliters of aqueous solution containing 60 micrograms of EGFP cDNA and shaked gently. Final cDNA concentration was 60 microgram/200 microliter.

### Composition IX:

Stock solution: dissolve 0.2 g PL in 2.5 g ethanol, add 6.3 g DDW in aliquots by mixing (Heidolph digital 2000 RZR-2000). Before the beginning of experiment (15 min) 180 microliters of stock solution were added in aliquots to 20 microliters of aqueous solution containing 60 micrograms p53 cDNA and shaked gently. Final cDNA concentration was 60 microgram/200 microliter.

### Cell Culture:

Subconfluent Osteosarcoma South cells were grown in DMEM on coverslips (five in each Petri dish of 5 cm in diameter).

### Transfection Method

Cells were washed twice with PBS, 2 ml of DMEM was added to each plate (containing 5 coverslips). 100 microliters of Compositions VI, VII, VIII or IX were added to the plates and the plates were incubated for 30 min at 37° C. Then, 2 ml of 20% Fetal Calf Serum (FCS) was added into each plate and incubation was continued for 24 h. Following incubation, the medium was removed, plates were washed twice with cold PBS, the cells fixed by adding 4 ml of methanol, and kept at −20° C. for at least 1 h. Cells were washed in PBS twice, left to rehydrate for at least 10 min. To detect GFP expression the coverslips were observed under a CLS microscope. The following parameters were set up before the experiment: pinhole size, electron gain, neutral density filters and background levels. In order to determine p53 expression, p53 immunostaining was performed with primary (anti p53 1801+DO-1) and secondary (antimouse Cy-3) antibodies and the coverslips were observed under a CLS microscope.

### Experimental Results

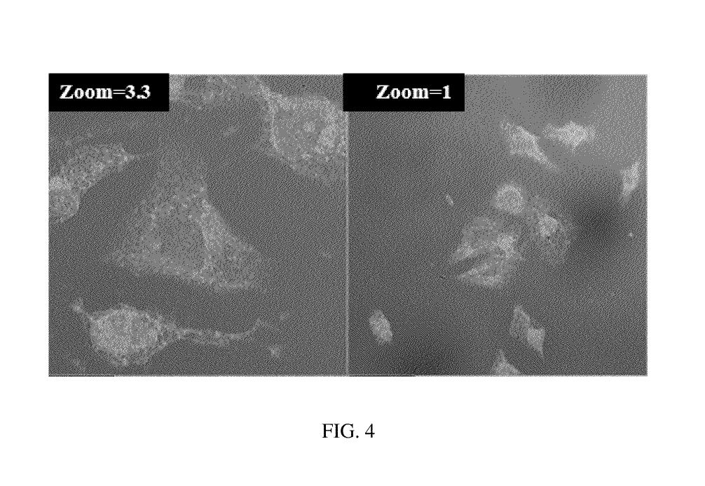

The results of this experiment are shown in FIG. 4. CLS micrograph demonstrates that cultured cells were efficiently transfected with p53 plasmid delivered from Composition VII.

Other Compositions with p53 Plasmid Containing Propranolol:

| | mg (% w/w) | | | | | | | |
|---|---|---|---|---|---|---|---|---|
| | A | | B | | C | | D | |
| PL | 0.8 mg | (2%) | 0.4 mg | (1%) | 2 mg | (5%) | 4 mg | (10%) |
| p53 cDNA | 10 μg | (0.025%) | 40 μg | (0.1%) | 10 μg | (0.025%) | 20 μg | (0.05%) |
| Propranolol | 40 μg | (0.1%) | 40 μg | (0.1%) | 80 μg | (0.2%) | 60 μg | (0.15%) |
| EtOH | 6 mg | (30%) | 5 mg | (20%) | 10 mg | (40%) | 20 mg | (50%) |
| DDW to | 40 mg | (100%) | 40 mg | (100%) | 40 mg | (100%) | 40 mg | (100%) |

Other Compositions with p53 Plasmid Containing THP:

| | mg (% w/w) | | | | | | | |
|---|---|---|---|---|---|---|---|---|
| | A | | B | | C | | D | |
| PL | 0.8 mg | (2%) | 0.4 mg | (1%) | 2 mg | (5%) | 4 mg | (10%) |
| p53 cDNA | 10 μg | (0.025%) | 40 μg | (0.1%) | 10 μg | (0.025%) | 20 μg | (0.05%) |
| THP | 40 μg | (0.1%) | 40 μg | (0.1%) | 80 μg | (0.2%) | 60 μg | (0.15%) |
| EtOH | 6 mg | (30%) | 5 mg | (20%) | 10 mg | (40%) | 20 mg | (50%) |
| DDW to | 40 mg | (100%) | 40 mg | (100%) | 40 mg | (100%) | 40 mg | (100%) |

### Example 5

### Intracellular Gene Delivery In Vivo

### Composition X:

Stock solution: 0.2 g PL were dissolved in 3 g ethanol and than 4.3 g DDW added in aliquots by mixing (Heidolph digital 2000 RZR-2000). The preparation was passed through antibacterial 0.2 μm filter. Before the beginning of experiment (15 min) 60 microliters of stock solution were added to 20 microliters of DDW containing 20 micrograms CMV-GFP cDNA in aliquots and the preparation was shaked gently. Final cDNA concentration was 2.5 microgram/10 microliter.

### Composition XI:

Stock solution: 0.2 g PL were dissolved in 3 g ethanol and than 4.3 g DDW was added in aliquots by mixing (Heidolph digital 2000 RZR-2000). The preparation was passed through antibacterial 0.2 μm filter. Before the beginning of the experiment (15 min) 60 microliters of stock solution were added to 20 microliters of DDW containing 40 micrograms CMV-GFP cDNA in aliquots and the preparation was shaked gently. Final cDNA concentration was 5 microgram/10 microliter.

### Composition XII:

Stock solution: 0.2 g PL were dissolved in 3 g ethanol and than 4.3 g DDW was added in aliquots by mixing (Heidolph digital 2000 RZR-2000). The preparation was passed through antibacterial 0.2 μm filter. Before the beginning of experiment (15 min) 60 microliters of stock solution was added to 20 microliters of DDW containing 200 micrograms CMV-GFP cDNA in aliquots and the preparation was shaked gently. Final cDNA concentration is 25 microgram/10 microliter.

Other Compositions with CMV-GFP cDNA Containing Propranolol:

| | mg (% w/w) | | | | | | | |
|---|---|---|---|---|---|---|---|---|
| | A | | B | | C | | D | |
| PL | 0.8 mg | (2%) | 0.4 mg | (1%) | 2 mg | (5%) | 4 mg | (10%) |
| CMV-GFP cDNA | 10 μg | (0.025%) | 40 μg | (0.1%) | 10 μg | (0.025%) | 20 μg | (0.05%) |
| Propranolol | 40 μg | (0.1%) | 40 μg | (0.1%) | 80 μg | (0.2%) | 60 μg | (0.15%) |
| EtOH | 6 mg | (30%) | 5 mg | (20%) | 10 mg | (40%) | 20 mg | (50%) |
| DDW to | 40 mg | (100%) | 40 mg | (100%) | 40 mg | (100%) | 40 mg | (100%) |

Other Compositions with CMV-GFP cDNA Containing THP:

| | mg (% w/w) | | | | | | | |
|---|---|---|---|---|---|---|---|---|
| | A | | B | | C | | D | |
| PL | 0.8 mg | (2%) | 0.4 mg | (1%) | 2 mg | (5%) | 4 mg | (10%) |
| CMV-GFP cDNA | 10 μg | (0.025%) | 40 μg | (0.1%) | 10 μg | (0.025%) | 20 μg | (0.05%) |
| THP | 40 μg | (0.1%) | 40 μg | (0.1%) | 80 μg | (0.2%) | 60 μg | (0.15%) |
| EtOH | 6 mg | (30%) | 5 mg | (20%) | 10 mg | (40%) | 20 mg | (50%) |
| DDW to | 40 mg | (100%) | 40 mg | (100%) | 40 mg | (100%) | 40 mg | (100%) |

### Application Method:

20, 40 or 60 microliters of Compositions X, XI, XII, Compositions containing propranolol or Compositions containing THP were applied to the dorsal skin surface of 5 week, female, CD-1 nude mice. The application area was covered with Hill Top patch. The bandage was removed within 48 hours. The animals were sacrificed after 3 weeks. The treated skin was removed and formation of GFP (green fluorescent protein) following intracellular gene delivery in whole tissue was visualized by CLSM as described.

### Experimental Results

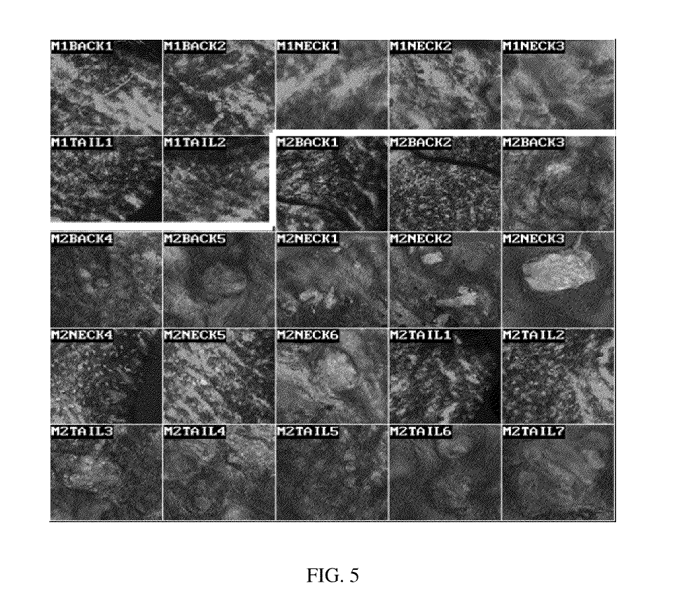

CLS micrograph demonstrates GFP intracellular expression, following transfection of whole tissue (skin) with CMV-GFP cDNA delivered from Composition VIII (FIG. 5).

1-20. (canceled)

21. A method for delivering a nucleic acid into a cell, the method comprising administering a composition comprising from 0.5 w/w to 10% w/w phospholipid, from 10% w/w to 50% w/w of one or more C2-C4 volatile alcohols, water and the nucleic acid, wherein the administration results in the intracellular delivery of the nucleic acid into the cell.

22. The method of claim 1, wherein the one or more C2-C4 volatile alcohols is ethanol.

23. The method of claim 1, wherein the nucleic acid comprises a nucleic acid sequence, DNA, RNA, nuclear RNA, mRNA, cDNA, genomic DNA, plasmid DNA, plasmids, or any combination thereof.

24. The method of claim 1, wherein the nucleic acid is a DNA oligonucleotide.

25. The method of claim 1, wherein the nucleic acid is a RNA oligonucleotide.

26. The method of claim 1, wherein the nucleic acid is complexed with the composition.

27. The method of claim 1, comprising contacting the composition with mammalian cells to transfect the mammalian cells.

28. The method of claim 1, comprising contacting the composition with plant cells to transfect the plant cells.

29. The method of claim 1, wherein the composition further comprises from 0.05 w/w to 3% w/w of at least one non-phospholipid cationic compound having a molecular weight ranging from about 100 grams/mole to about 600 grams/mole.

30. The method of claim 1, wherein the nucleic acid is intracellularly delivered via intradermal administration.

31. The method of claim 1, wherein the nucleic acid is intracellularly delivered via intraperitoneal (IP) administration.

32. The method of claim 1, wherein the nucleic acid is intracellularly delivered via intramuscular (IM) administration.

33. The method of claim 1, wherein the nucleic acid is intracellularly delivered via subcutaneous (SC) administration.

34. The method of claim 1, wherein the nucleic acid is intracellularly delivered via intravenous (IV) administration.

35. The method of claim 1, wherein the nucleic acid is intracellularly delivered via intratumoral administration.

36. The method of claim 1, wherein the nucleic acid is intracellularly delivered via intradermal administration.

37. The method of claim 1, wherein the nucleic acid is intracellularly delivered via iontophoresis.

38. The method of claim 1, wherein the nucleic acid is intracellularly delivered via phonophoresis.

39. The method of claim 1, wherein the nucleic acid is intracellularly delivered via microporation.

40. The method of claim 1, wherein the nucleic acid is intracellularly delivered via microneedles.

41. The method of claim 1, wherein the nucleic acid is intracellularly delivered via jet laser administration.

42. The method of claim 1, wherein the nucleic acid is intracellularly delivered via the skin.
